(19) United States
(12) Patent Application Publication
(10) Pub. No.: US 2024/0050650 A1
(43) Pub. Date: Feb. 15, 2024

(54) AMBULATORY INFUSION PUMP SYSTEM INCLUDING PUMP HOLDER

(71) Applicant: Tandem Diabetes Care, Inc., San Diego, CA (US)

(72) Inventors: Adam Evard, San Diego, CA (US); Jacob Pearlman, San Diego, CA (US); Virginia S. Lu, San Diego, CA (US); Neel Shah, San Diego, CA (US)

(21) Appl. No.: 18/448,584

(22) Filed: Aug. 11, 2023

Related U.S. Application Data
(60) Provisional application No. 63/397,234, filed on Aug. 11, 2022.

(51) Int. Cl. A61M 5/142

(52) U.S. Cl. CPC A61M 5/14248

(57) ABSTRACT

Embodiments of the present disclosure relate to an infusion pump system including a holder or carrier for an ambulatory infusion pump that can be worn on or near the patient's body. Embodiments depicted and described herein provide a more versatile and secure way to hold and carry an ambulatory infusion pump.

### PRIORITY CLAIM

The present application claims the benefit of U.S. Provisional Patent Application No. 63/397,234 filed Aug. 11, 2022, which is hereby incorporated herein by reference.

### TECHNICAL FIELD

The present disclosure relates to medical pumps for delivering medicament to a patient, and more specifically, to an ambulatory infusion pump that can be worn on the body of a user.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type I, or in some cases, type II diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps may be worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

One type of pump that has been developed is a patch pump also known as a micro pump. Patch pumps are small pumps, typically ambulatory, that may be carried directly on the skin under the user's clothing. In some cases, the pumps are situated directly on, or very near to, the injection site such that little or no tubing is required to deliver the insulin or other medicament to the patient. These pumps can therefore be worn directly on the body of the user or carried in close proximity to the body. Typically, patch pumps that are worn on the body are affixed to the body with an adhesive patch. Some patch pumps include a single button on the pump to initiate delivery of medicament and do not include a built-in display or user interface. These pumps are therefore primarily remote-controlled, which enables programming of the devices without removing them from the body.

### SUMMARY

Embodiments of the present disclosure relate to an infusion pump system including a holder or carrier for an ambulatory infusion pump that can be worn on or near the patient's body. Embodiments depicted and described herein provide a more versatile and secure way to hold and carry an ambulatory infusion pump.

In embodiments, an ambulatory infusion pump system can include an ambulatory infusion pump configured to contain a medicament and including a switch disposed on a perimeter of the infusion pump that is movable between a first position and a second position. A pump holder can be configured to releasably hold the infusion pump and include an adhesive patch configured to retain the pump holder on a body of a user. The pump holder can include a frame base having a shape generally matching the perimeter of the infusion pump and a locking projection extending from the frame base and configured to interface with the switch disposed on the infusion pump. When the switch is moved from the first position to the second position with the infusion pump disposed on the pump holder, the infusion pump is releasably locked on the frame base.

In embodiments, a pump holder for an ambulatory infusion pump can include an adhesive patch configured to retain the pump holder on a body of a user, a frame base having a shape generally matching a perimeter of an ambulatory infusion pump and a locking projection extending from the frame base and configured to interface with a switch disposed on the infusion pump. When the switch is moved from the first position to the second position with the infusion pump disposed on the pump holder, the infusion pump is releasably locked on the frame base.

In embodiments, an ambulatory infusion pump configured to contain a medicament can include a switch disposed on a perimeter of the infusion pump that is movable between a first position and a second position. When the infusion pump is disposed on a pump holder, the infusion pump is releasably locked on the pump holder when the switch is moved from the first position to the second position causing the switch to interface with a locking projection on the pump holder.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1C depicts an embodiment of a pump system according to the disclosure.

FIGS. 2A-2B depict an embodiment of a pump system according to the disclosure.

FIGS. 3A-3D depict an embodiment of a pump system according to the disclosure.

FIGS. 4A-4C depict the pump system of FIGS. 3A-3D.

FIGS. 5A-5B depict the pump system of FIGS. 3A-3D.

FIG. 6 depicts an embodiment of a pump system according to the disclosure.

FIGS. 7A-7B depict remote control devices for a pump system according to embodiments of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

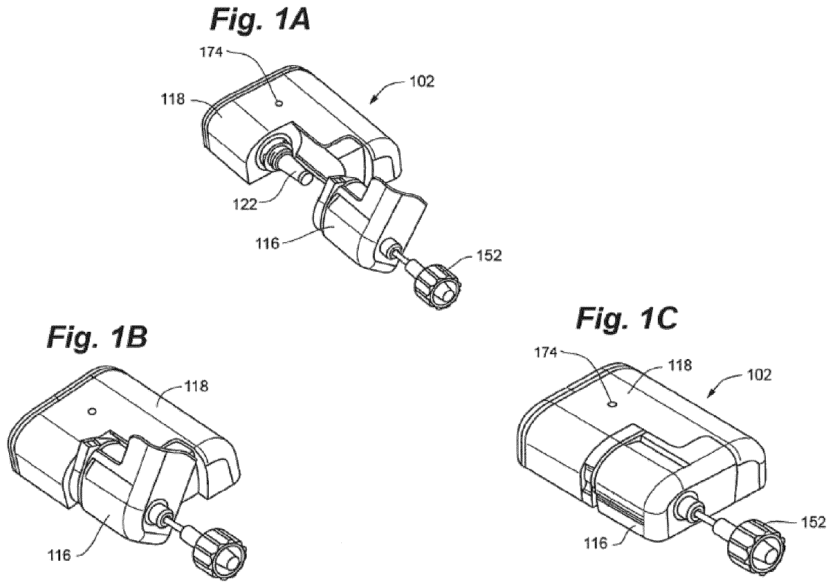

Referring to FIGS. 1A-1C, a pump system 100 including a pump 102 is depicted in accordance with an embodiment of the disclosure. Drive unit 118 of pump 102 includes a drive mechanism 122 that mates with a recess in medicament cartridge 116 of pump 102 to attach the medicament cartridge 116 to the drive unit 118. Further details regarding example embodiments of such delivery mechanisms can be found in U.S. Patent Publication No. 2017/0049957, which is hereby incorporated by reference in its entirety. In some embodiments, cartridge 116 can rotationally attach to drive unit 118 as shown in FIGS. 1B-1C. Further details regarding such rotational attachment can be found in, for example, U.S. Pat. No. 9,993,595, which is hereby incorporated by reference in its entirety.

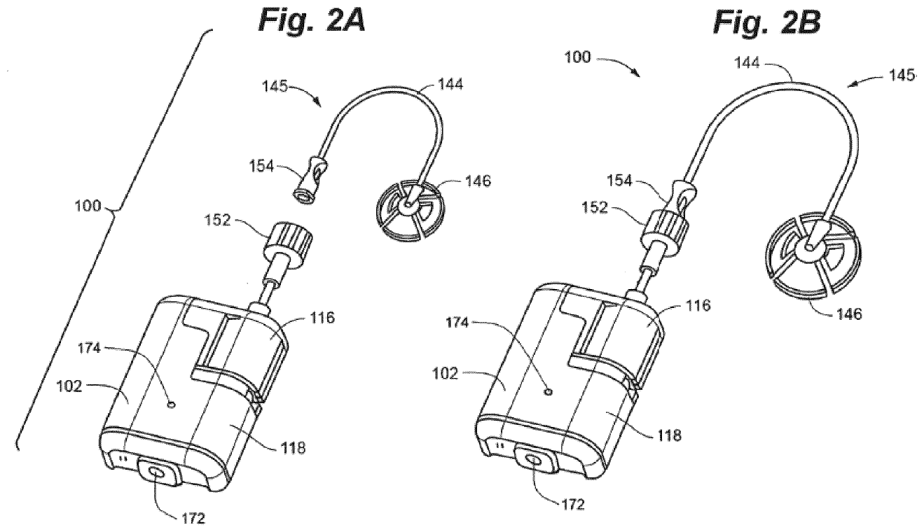

As depicted in the embodiment of FIGS. 2A-2B, pump system 100 can include a pump 102 and an infusion set 145. FIG. 2A depicts this infusion set 145 as not connected to pump while FIG. 2B depicts infusion set 145 connected to pump 102 via connectors 154 and 152. Infusion set 145 can include tubing 144 extending between a connector 154 and a site connector 146. Connector 154 can be configured to couple to pump 102 at connector 152. Site connector 146 can be configured to be attached to an infusion site on a user, while pump 102 can be carried in a separate location, such as the user's pocket or another location on the user's body. Various lengths of tubing 144 can be used in this embodiment to accommodate the user's preference. Further details regarding such pumps can be found in U.S. Pat. Nos. 9,993,595; 10,279,106; and 10,279,107, each of which is hereby incorporated herein by reference in its entirety.

In embodiments, pump 102 includes a processor that controls operations of the pump and, in some embodiments, may communicate in either one-way or two-way modes to, e.g., receive operational commands and/or other signals, including data, from a separate device and/or, e.g., to send signals, including data, to a separate device. Pump 102 can include one or more buttons configured to cause the processor to initiate one or more functions. In the depicted embodiment, pump 102 includes only a single button 172, although more than one button may be present on pump 102. Button 172 can be configured to, for example, initiate delivery of medicament. Any single button such as button 172 can be utilized to execute a plurality of functions or operations. For example, a single press of button may initiate one function, holding the button down for a predetermined period of time may initiate another function, etc. Because the depicted pump 102 optionally does not itself include a display or user interface, information and feedback regarding medicament delivery or dosing initiated with button 172 can be communicated to and displayed on a remote control device or other device having a display and/or other type of user interface.

In embodiments, pump 102 includes a light source, such as a light emitting diode (LED) 174. Light source 174 can be configured to provide user feedback regarding user input and/or the performance of a desired function. For example, light source 174 can illuminate or blink one or more times to indicate that the one or more buttons 172 have been activated and/or that a desired function has been initiated. In embodiments, pump 102 can additionally and/or alternatively vibrate and/or provide audible notifications to indicate that the one or more buttons 172 have been activated and/or that a desired function has been initiated or, e.g., to provide user feedback regarding user input and/or the performance of the desired function. Illumination of light source 174 and/or vibrations and/or audible notifications may be executed in any number of patterns, frequencies, durations, sequences, combinations, colors, brightness levels, etc. to indicate particular information, such as particular input received and/or particular functions or operations enabled and/or initiated, to the pump user or caregiver. In some embodiments pump can include two or more indicator lights 174.

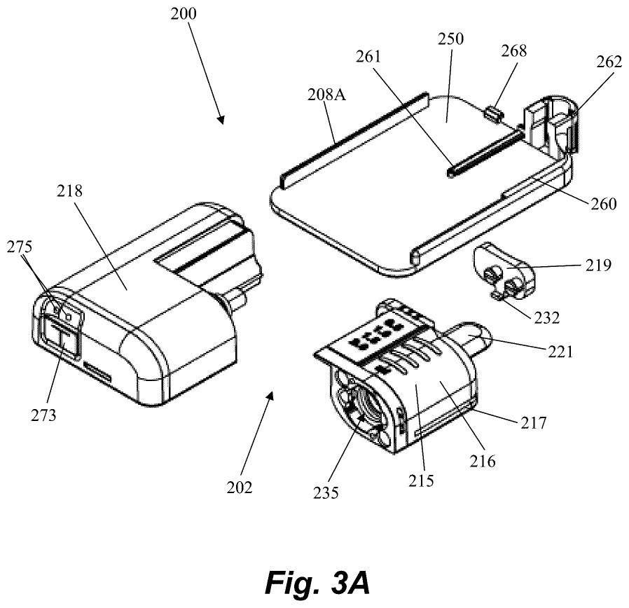

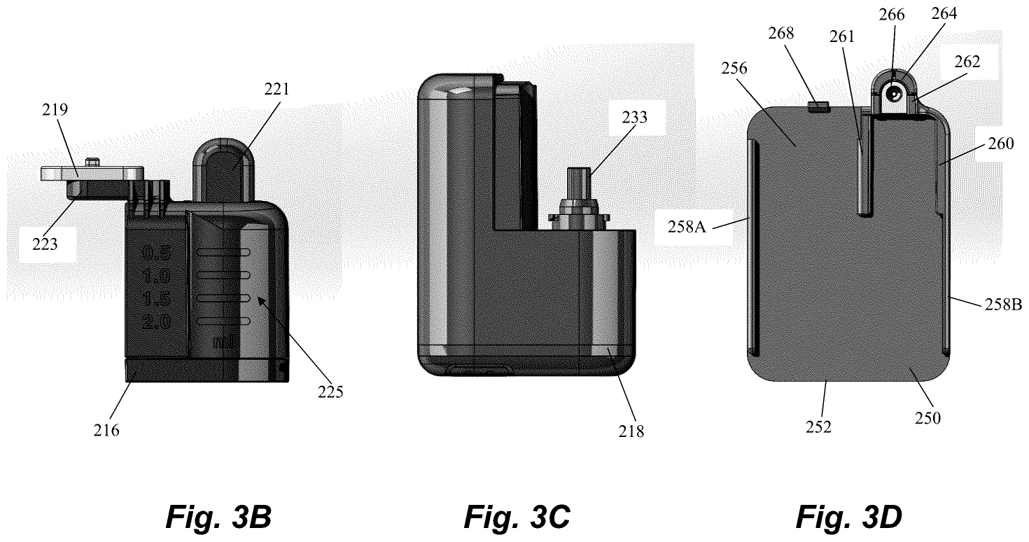

As noted above, in some embodiments pump 102 can be provided with a connector 152 extending from tubing attached to the pump 102 to an infusion set 145 for delivery of medicament at a site displaced from the pump 102. In other embodiments, pump 102 can be configured to deliver medicament through a cannula extending directly beneath the pump 102. One example of a pump system 200 capable of delivering medicament from a cartridge 216 in this manner is depicted in FIGS. 3A-3D. In some embodiments, a common drive unit can be used interchangeably with a cartridge 116 having tubing and a connector 152 for use with an infusion set 152 and a cartridge 216 configured to deliver medicament through a cannula directly below the cartridge 216 to meet the needs of the user at a given time.

Referring now to FIGS. 3A-3D, an infusion pump system 200 including a user-wearable infusion pump 202 including a cartridge 216 capable of delivering medicament through a cannula directly beneath the pump is depicted. Cartridge 216 can, in some embodiments, rotationally attach to a drive unit 218 that includes an input button 273 and a pair of indicator lights 275. Cartridge 216 can be at least partially comprised of a transparent material and include graduated markings 225 to enable a user to visually determine an amount of medicament in the cartridge reservoir 215. Cartridge can further include a cannula interface 221 having an outlet fluidly connected to the interior of the reservoir 215 and configured to interface with a cannula extending below the pump to deliver medicament from the reservoir 215 to the user. A clip or switch 219 can be disposed on cartridge 216 and slide along a switch mount 223 on cartridge to releasably secure the pump 202 to a pump holder 250, as will be described in more detail below. In some embodiments, cartridge 216 is configured to be refilled while in other embodiments the cartridge 216 is disposable and discarded after a single use to be replaced with a new cartridge. If the cartridge is disposable, one advantage of providing the switch 219 on the cartridge is that if the switch is broken or otherwise damaged a new cartridge can be attached to the re-usable drive unit 218 with a functioning switch 219. Additionally or alternatively, the switch 219 can be selectively detachable from the cartridge 216 such that if the switch 219 is damaged, it can be removed and a new switch attached.

Drive unit can include a cartridge attachment projection 233 configured to receive an opening 235 in the cartridge 218 for rotational attachment of the cartridge 216 to the drive unit 218. In operation, a drive screw can extend from drive unit 218 and into cartridge 216 with a plunger disposed within reservoir 215 of cartridge 216 such that advancement of the lead screw by the drive unit 218 causes the plunger to advance within the reservoir 215 to incrementally dispense medicament from the reservoir 215.

Infusion pump system 200 can further include a pump holder 250 that can be used to releasably contain a user-wearable infusion pump 202. Holder 250 can include a frame 252 disposed on an adhesive patch (not pictured) configured to attach the pump holder 200 directly to the body of a user. Frame 252 can include a base 256 on which the pump rests and one or more perimeter walls 258A, 258B that surround one or more portions of the perimeter of a pump disposed on base 256. Any of the perimeter walls 258 can include engagement features that aid in retaining pump on holder 250. For example, in the depicted embodiment perimeter wall 258B includes an inwardly extending locking projection 260. When the pump 202 is disposed on the holder 250, a corresponding projection 217 on the cartridge 216 nests beneath the locking projection 260 to aid in retaining the pump 202 on the holder 250. Pump holder 250 can further include a guide rail 261 configured to interface with a corresponding recess in pump 202 to aid in proper alignment of pump 202 on holder 250, as will be further discussed below, and to further aid in retention of pump. Pump holder 250 can also be provided with a locking projection 268 that interfaces with switch 219 on pump to releasably lock the pump 202 on the holder 250, as will also be discussed in more detail below.

Pump holder 250 can include a cannula/septum module 262. Cannula/septum module can include a module frame 264 sized to receive the cannula interface 221 of the cartridge 216 and a cannula aperture 266 configured to fluidly connect the outlet of the cartridge 216 with a cannula extending through cannula/septum module 262 beneath the pump holder 200 and into the user's skin. In practice, a needle can be inserted through the cannula aperture 266 to insert the cannula into the skin and then removed prior to inserting the pump 202 onto the pump holder 250, leaving the cannula in place.

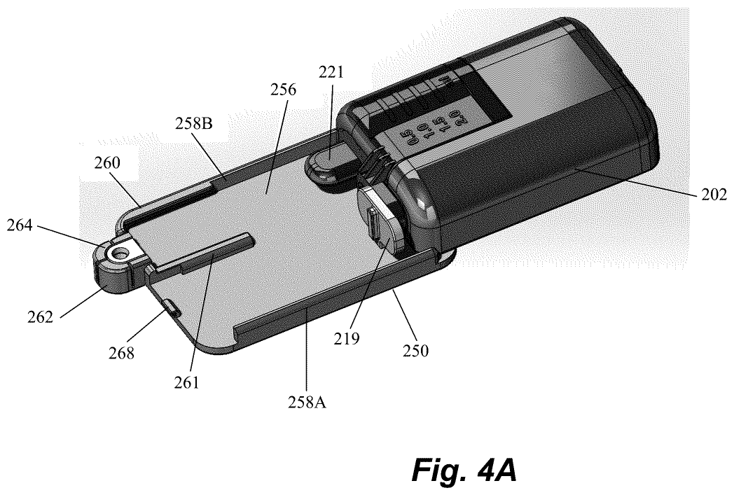

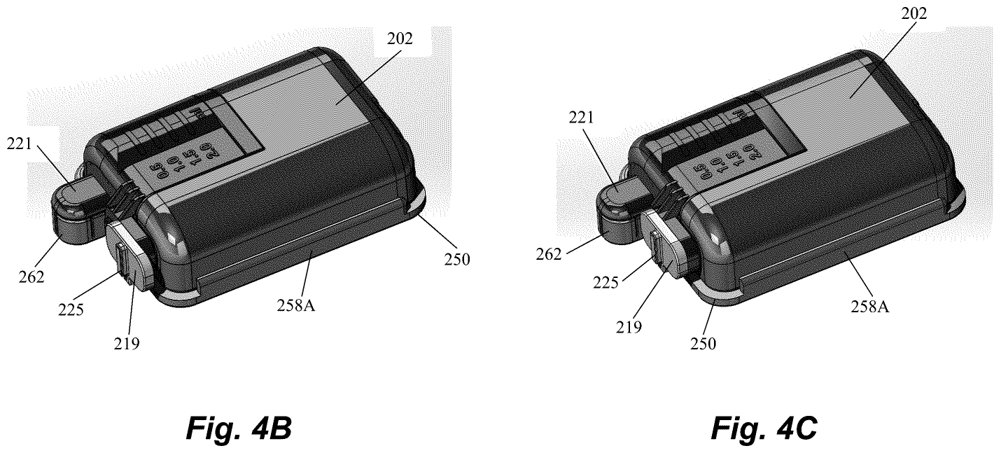

Referring now to FIGS. 4A-4C, pump 202 can be inserted onto pump holder 250 by first positioning the perimeter of pump between perimeter walls 258A, 258B as depicted in FIG. 4A. The pump 202 can then be slid along the frame base 256 such that the guide rail 261 on the holder 250 interfaces with the corresponding feature on the bottom side of the pump and the projection 217 on pump slides beneath the locking projection 260 of retention wall 258B until the cannula interface 221 on pump is mated with the module frame 264 of the septum/cannula module 262 of the holder, as shown in FIG. 4B. To releasably lock the pump 202 on the holder 250, the switch 219 on cartridge 216 is then slid laterally along the switch mount 223 (towards the cannula interface 221) from an unlocked position (FIG. 4B) to a locked position (FIG. 4C). In some embodiments, switch 219 can include a switch grip 225 that aids the user in grasping and sliding the switch 219.

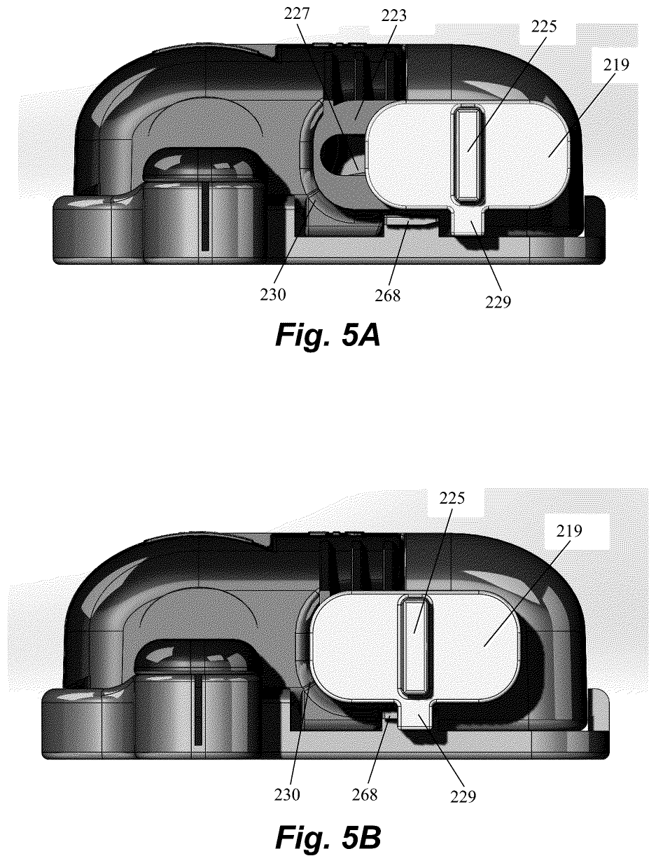

Referring now to FIG. 5A-5B, further details regarding the unlocked (FIG. 5A) and locked (FIG. 5B) positions of the switch 219 are depicted. Upon initial insertion of pump 202 onto holder 250, a switch lock 229 extending downwardly from switch 219 is offset from the locking projection 268 on holder. Switch 219 can then be slid laterally along a slot 227 in switch mount 223 on pump 202 to align the switch lock 229 with the locking projection 268. The cartridge 216 of pump 202 can define a switch stop 230 that stops movement of the switch 219 to ensure that the switch 219 is properly aligned with the locking projection 268. In addition to the portion of switch lock 229 that extends downwardly from switch 219, switch lock 229 can also include an inward projection 232 (see FIG. 3A) that nests beneath the locking projection 268. As such, in the locked position the switch 219 inhibits movement both of the pump both laterally back off of the frame (i.e., into the page) and vertically upwardly off of the frame. When the pump 202 needs to be removed from the holder 250, the switch 219 can be slid back to the first, unlocked position and the pump 202 can be slid back off of the holder 250.

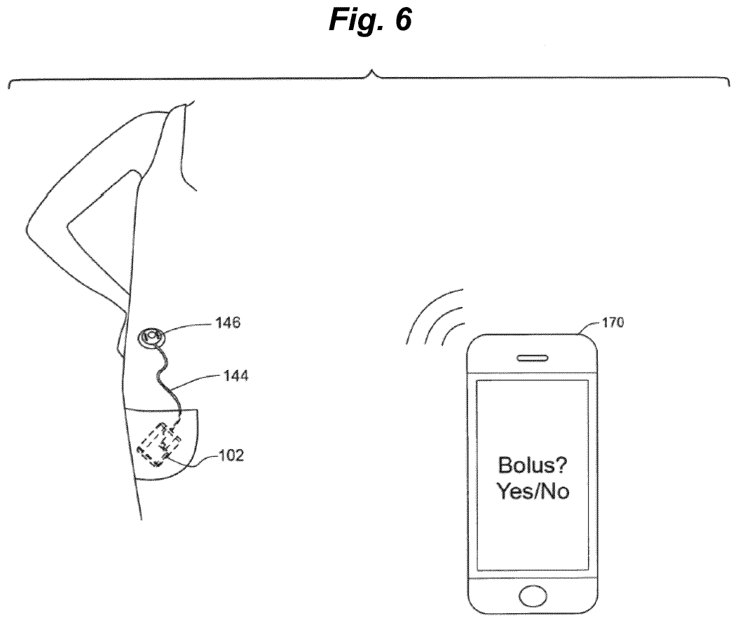

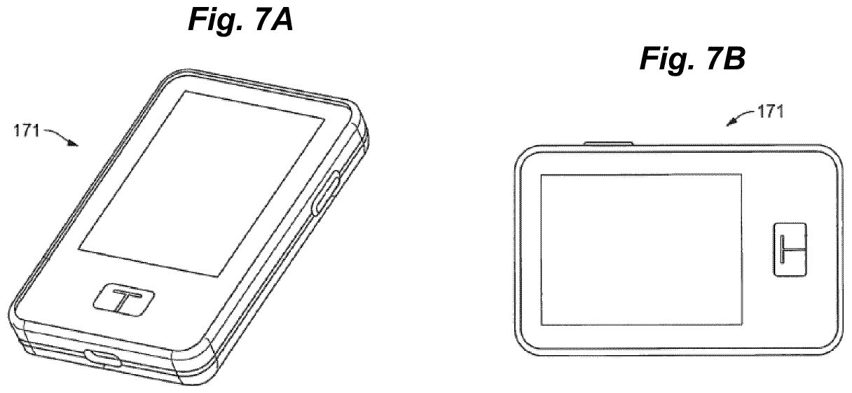

Referring to FIGS. 6-7B, one or more remote control devices 170, 171 can be used to communicate with the processor of pumps as disclosed herein to control delivery of medicament and transfer data with pump via a wired or a wireless electromagnetic signal, such as via, e.g., a near field communication (NFC) radio frequency (RF) modality or other RF modalities such as Bluetooth®, Bluetooth® low energy, mobile or Wi-Fi communication protocols, for example, according to embodiments of the present disclosure. Such a remote control can include, for example, a mobile communication device 170, such as a smart phone (as depicted in FIG. 6) executing a software application for control of the pump, a dedicated remote controller 171 (as depicted in FIGS. 7A-7B), a wearable electronic watch or electronic health or fitness monitor or personal digital assistant (PDA), etc., or a tablet, laptop or personal computer. Such communications between (and among) the one or more remote control devices 170, 171 and pump may be one-way or two-way for, e.g., effective transfer of data among the devices and the pump, control of pump operations, updating software on the devices and/or pump, and allowing pump-related data to be viewed on the devices and/or pump. Although pump 102 is shown in FIG. 6 as being carried by the user with an infusion set 144, 146 extending to the user's body, it should be understood that pumps worn directly on the body such as those described herein may also be controlled and communicate in such a manner.

Although the embodiments herein have been specifically described with respect to a user-wearable infusion pump, the inventions disclosed herein could be employed with any other type of medical device capable of being worn on or near the body. Embodiments could further include non-medical applications, including smartphones. For example, a smartphone or other device could be placed into carriers such as those described herein to be interchangeably attached to various accessories.

In embodiments, an ambulatory infusion pump system can include an ambulatory infusion pump configured to contain a medicament and including a switch disposed on a perimeter of the infusion pump that is movable between a first position and a second position. A pump holder can be configured to releasably hold the infusion pump and include an adhesive patch configured to retain the pump holder on a body of a user. The pump holder can include a frame base having a shape generally matching the perimeter of the infusion pump and a locking projection extending from the frame base and configured to interface with the switch disposed on the infusion pump. When the switch is moved from the first position to the second position with the infusion pump disposed on the pump holder, the infusion pump is releasably locked on the frame base.

In some embodiments, the infusion pump includes a drive unit and a cartridge including a reservoir configured to contain the medicament selectively attachable to the drive unit, and the switch is disposed on the cartridge.

In some embodiments, the switch is mounted in an elongate slot in the infusion pump, and the switch is laterally slidable within the elongate slot between the first position and the second position.

In some embodiments, in the second position the switch is aligned with the locking projection to prevent the infusion pump from being slid back laterally off of the frame base.

In some embodiments, the switch further includes an inwardly extending projection configured to nest beneath the locking projection when the switch is in the second position, with the inwardly extending projection configured to prevent the infusion pump from being removed vertically off of the frame base.

In some embodiments, the pump holder includes an upwardly extending guiderail and the infusion pump includes an elongate recess on a bottom surface of the infusion pump, and the guiderail is configured to be inserted into the recess to guide the infusion pump onto the pump holder.

In some embodiments, the pump holder further comprises one or more perimeter walls extending upwardly from an outer perimeter of the frame base.

In some embodiments, at least one perimeter wall includes a locking projection extending inwardly towards the frame base, and a portion of the infusion pump is configured to nest beneath the locking projection to aid in retaining the infusion pump on the pump holder.

In some embodiments, the portion of the infusion pump configured to next beneath the locking projection is a projection extending outwardly from the infusion pump.

In some embodiments, the pump holder further comprises a cannula interface through which a cannula is configured to be inserted for delivery of the medicament from the infusion pump directly beneath the pump holder.

In embodiments, a pump holder for an ambulatory infusion pump can include an adhesive patch configured to retain the pump holder on a body of a user, a frame base having a shape generally matching a perimeter of an ambulatory infusion pump and a locking projection extending from the frame base and configured to interface with a switch disposed on the infusion pump. When the switch is moved from the first position to the second position with the infusion pump disposed on the pump holder, the infusion pump is releasably locked on the frame base.

In some embodiments, in the second position the locking projection is configured to be aligned with the switch to prevent the infusion pump from being slid back laterally off of the frame base.

In some embodiments, there is a recess beneath the locking projection and an inwardly extending projection of the switch is configured to nest within the recess beneath the locking projection when the switch is in the second position to prevent the infusion pump from being removed vertically off of the frame base.

In some embodiments, the pump holder includes a guiderail extending upwardly from the frame base configured to interface with a recess in the infusion pump to guide the infusion pump onto the pump holder.

In some embodiments, the pump holder further comprises one or more perimeter walls extending upwardly from an outer perimeter of the frame base.

In some embodiments, at least one perimeter wall includes a locking projection extending inwardly towards the frame base, and a portion of the infusion pump is configured to nest beneath the locking projection to aid in retaining the infusion pump on the pump holder.

In some embodiments, the pump holder further comprises a cannula interface through which a cannula is configured to be inserted for delivery of medicament from the infusion pump directly beneath the pump holder.

In embodiments, an ambulatory infusion pump configured to contain a medicament can include a switch disposed on a perimeter of the infusion pump that is movable between a first position and a second position. When the infusion pump is disposed on a pump holder, the infusion pump is releasably locked on the pump holder when the switch is moved from the first position to the second position causing the switch to interface with a locking projection on the pump holder.

In some embodiments, the infusion pump includes a drive unit and a cartridge including a reservoir configured to contain the medicament selectively attachable to the drive unit, and the switch is disposed on the cartridge.

In some embodiments, the switch is mounted in an elongate slot in the infusion pump, and the switch is laterally slidable within the elongate slot between the first position and the second position.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patent application Ser. Nos. 17/368,968; 17/896,492; 18/011,060; 18/071,814; 18/071,835; 18/075,029; 18/090,788; 18/115,316; 18/139,391 and 18/220,156.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. An ambulatory infusion pump system, comprising:
an ambulatory infusion pump configured to contain a medicament, the infusion pump including a switch disposed on a perimeter of the infusion pump, the switch movable between a first position and a second position;
a pump holder configured to releasably hold the infusion pump and including an adhesive patch configured to retain the pump holder on a body of a user, the pump holder comprising:
a frame base having a shape generally matching the perimeter of the infusion pump; and
a locking projection extending from the frame base and configured to interface with the switch disposed on the infusion pump, wherein when the switch is moved from the first position to the second position with the infusion pump disposed on the pump holder, the infusion pump is releasably locked on the frame base.

2. The ambulatory infusion pump system of claim 1, wherein the infusion pump includes a drive unit and a cartridge including a reservoir configured to contain the medicament selectively attachable to the drive unit, and wherein the switch is disposed on the cartridge.

3. The ambulatory infusion pump system of claim 1, wherein the switch is mounted in an elongate slot in the infusion pump, and wherein the switch is laterally slidable within the elongate slot between the first position and the second position.

4. The ambulatory infusion pump system of claim 1, wherein in the second position the switch is aligned with the locking projection to prevent the infusion pump from being slid back laterally off of the frame base.

5. The ambulatory infusion pump system of claim 4, wherein the switch further includes an inwardly extending projection configured to nest beneath the locking projection when the switch is in the second position, the inwardly extending projection configured to prevent the infusion pump from being removed vertically off of the frame base.

6. The ambulatory infusion pump system of claim 1, wherein the pump holder includes an upwardly extending guiderail and the infusion pump includes an elongate recess on a bottom surface of the infusion pump, and wherein the guiderail is configured to be inserted into the recess to guide the infusion pump onto the pump holder.

7. The ambulatory infusion pump system of claim 1, wherein the pump holder further comprises one or more perimeter walls extending upwardly from an outer perimeter of the frame base.

8. The ambulatory infusion pump system of claim 7, where at least one perimeter wall includes a locking projection extending inwardly towards the frame base, and wherein a portion of the infusion pump is configured to nest beneath the locking projection to aid in retaining the infusion pump on the pump holder.

9. The ambulatory infusion pump system of claim 4, wherein the portion of the infusion pump configured to next beneath the locking projection is a projection extending outwardly from the infusion pump.

10. The ambulatory infusion pump system of claim 1, wherein the pump holder further comprises a cannula interface through which a cannula is configured to be inserted for delivery of the medicament from the infusion pump directly beneath the pump holder.

11. A pump holder for an ambulatory infusion pump, comprising:
an adhesive patch configured to retain the pump holder on a body of a user;
a frame base having a shape generally matching a perimeter of an ambulatory infusion pump; and
a locking projection extending from the frame base and configured to interface with a switch disposed on the infusion pump, wherein when the switch is moved from the first position to the second position with the infusion pump disposed on the pump holder, the infusion pump is releasably locked on the frame base.

12. The pump holder of claim 1, wherein in the second position the locking projection is configured to be aligned with the switch to prevent the infusion pump from being slid back laterally off of the frame base.

13. The pump holder of claim 12, wherein further comprising a recess beneath the locking projection and wherein an inwardly extending projection of the switch is configured to nest within the recess beneath the locking projection when the switch is in the second position to prevent the infusion pump from being removed vertically off of the frame base.

14. The pump holder of claim 11, wherein the pump holder includes a guiderail extending upwardly from the frame base configured to interface with a recess in the infusion pump to guide the infusion pump onto the pump holder.

15. The pump holder of claim 11, wherein the pump holder further comprises one or more perimeter walls extending upwardly from an outer perimeter of the frame base.

16. The pump holder of claim 15, where at least one perimeter wall includes a locking projection extending inwardly towards the frame base, and wherein a portion of the infusion pump is configured to nest beneath the locking projection to aid in retaining the infusion pump on the pump holder.

17. The ambulatory infusion pump system of claim 11, wherein the pump holder further comprises a cannula interface through which a cannula is configured to be inserted for delivery of medicament from the infusion pump directly beneath the pump holder.

18. An ambulatory infusion pump configured to contain a medicament, the infusion pump including a switch disposed on a perimeter of the infusion pump, the switch movable between a first position and a second position,
wherein when the infusion pump is disposed on a pump holder, the infusion pump is releasably locked on the pump holder when the switch is moved from the first position to the second position causing the switch to interface with a locking projection on the pump holder.

19. The ambulatory infusion pump of claim 18, wherein the infusion pump includes a drive unit and a cartridge including a reservoir configured to contain the medicament selectively attachable to the drive unit, and wherein the switch is disposed on the cartridge.

20. The ambulatory infusion pump of claim 18, wherein the switch is mounted in an elongate slot in the infusion pump, and wherein the switch is laterally slidable within the elongate slot between the first position and the second position.
